(19) United States
(12) Patent Application Publication
(10) Pub. No.: US 2024/0335251 A1
(43) Pub. Date: Oct. 10, 2024

(54) AUTOMATIC RFID TOOL COUPLING

(71) Applicant: Aesculap AG, Tuttlingen (DE)

(72) Inventors: Frederick Lenzenhuber, Tuttlingen (DE); André Buerk, Villingen-Schwenningen (DE)

(21) Appl. No.: 18/579,472

(22) PCT Filed: Jul. 11, 2022
(86) PCT No.: PCT/EP2022/069312

§ 371 Date: Jan. 15, 2024

(30) Foreign Application Priority Data
Jul. 16, 2021 (DE) 10 2021 118 412.6

(51) Int. Cl. A61B 90/98; A61B 17/16

(52) U.S. Cl. CPC A61B 90/98; A61B 17/162; A61B 17/1622

(57) ABSTRACT

A medical tool has a tool shaft with a shaft geometry for introducing torque and for axial securement. The shaft geometry has at least one trough-shaped indentation in the outer lateral surface of the tool shaft.

### CROSS-SECTION TO RELATED APPLICATIONS

This application is the United States national stage entry of International Application No. PCT/EP2022/069312, filed on Jul. 11, 2022, and claims priority to German Application No. 10 2021 118 412.6, filed on Jul. 16, 2021. The contents of International Application No. PCT/EP2022/069312 and German Application No. 10 2021 118 412.6 are incorporated by reference herein in their entireties.

### FIELD

The present disclosure relates to a medical/surgical tool or instrument, in particular a tool identifiable via an identification element, which is provided with a shaft geometry that enables both axial securing and torque transmission to the tool.

### BACKGROUND

In medicine today, tools are already inserted whose working end can be recognized automatically. The working end represents the functional part of the tool and can be shaped in a variety of ways depending on the application. Tools of the type referred to herein include tools, instruments, spray nozzles, HF tips, ultrasonic blades and the like that are generally used in the field of medicine.

Such recognition of the working end is achieved via digitally readable data storages attached to the tool, which act as an identification element and enable the tool or the working end of the tool to be identified.

However, housing the digitally readable data storages on/in the tool poses problems for the industry. Medical tools are generally compact and must be able to withstand harsh operating conditions despite their delicate design. However, attaching a digitally readable data storage in/on a tool weakens the structure of the tool and may cause notch stresses in the tool. In order to counteract this, the tools and in particular the torque-transmitting portions of the tool are made more robust, which increases the dimensions of the tool and thus makes the tools more unwieldy to handle.

There is therefore a need for a way of housing a digitally readable data storage/identification element on/in a tool, whereby a large torque can be transmitted from a force application site/the shaft geometry to a working end despite the compact/delicate design of the tool.

### SUMMARY

The present disclosure describes a medical tool comprising a tool shaft that has, in a shaft region, a shaft geometry for introducing torque and axially securing. This shaft geometry for introducing torque and simultaneously axially securing consists solely of at least one (or several) trough-shaped or bathtub-shaped depression(s)/indentation(s)/latching bulges(s) in the outer lateral surface of the tool shaft.

In other words, the tool of the present disclosure has the shaft region on or along the tool shaft, wherein the shaft region is provided with a shaft geometry that enables both the torque to be transmitted to the tool and the tool to be axially secured. According to the invention, this is achieved solely/exclusively via the at least one trough-shaped indentation in the surface of the tool shaft in the shaft region.

The core of the disclosure is therefore that both the torque transmission and the axial securing of the tool are ensured solely via at least one trough-shaped depression in the lateral surface of the tool shaft. Accordingly, these two functions are not divided into two shaft regions/functional portions spaced apart in the longitudinal shaft direction.

By combining the axial securing and the torque transmission into just one (single) geometry, it is possible to make the tool more compact and still ensure high torque transmission from the torque introduction site in the form of the shaft geometry to the working end. In addition, the trough-shaped geometry of the shaft geometry prevents notch stresses on the tool shaft and increases the torsional rigidity of the tool shaft.

In a first preferred aspect, the shaft geometry of the tool consists of several trough-shaped depressions/latching bulges distributed evenly over the circumference of the tool.

In other words, the shaft geometry has more than one trough-shaped depression along a radial portion of the tool shaft, each trough-shaped depression having the same angular distance radially from its neighboring trough-shaped depression.

In yet other words, the distance in a circumferential direction from each trough-shaped depression to its neighboring trough-shaped depression is constant over the entire circumference of the tool.

Several trough-shaped depressions, evenly distributed over the circumference of the tool, ensure a uniform introduction of drive torque into the tool. This reduces the load on the tool and the load on the tool support.

In a further preferred aspect, the tool has an orientation portion arranged proximally to the shaft geometry, wherein the orientation portion preferably has a pyramid-shaped geometry. In particular, the pyramid tip of the orientation portion is located on the center fiber of the tool.

In other words, the orientation portion, which is preferably configured in a pyramid shape, is provided on the side of the shaft geometry opposite the working end of the tool. The pyramid tip lying on the central fiber of the tool forms the point of the tool furthest away from the working end.

In a further preferred aspect, a number of pyramid sides corresponds to a number of trough-shaped depressions.

In other words, the number of pyramid sides of the orientation portion of the tool is matched to the number of trough-shaped depressions of the shaft geometry of the tool. Preferably, the number of pyramid sides and the number of trough-shaped depressions is three.

In a further preferred aspect, the number of the trough-shaped indentations is an integer multiple of the number of pyramid sides.

In a further preferred aspect, the tool comprises an identification element which is arranged in a sleeve made of (one piece (of material)) containing the orientation portion.

In other words, at a proximal portion of the tool, the sleeve preferably made of one piece (of material) is attached to the tool, which is provided and configured to receive the identification element. The orientation portion is located at the proximal front side of the sleeve. The sleeve is preferably force-fittingly and/or form-fittingly connected to the tool shaft.

The identification element enables the individual tool to be recognized. This can be used to detect and document the actual service life of individual tools in combination with a handpiece. In a further step, it is thus possible to reliably determine the service life of the tools. In addition, tool-related (use) parameters can be stored on the identification element, which are then preferably automatically transferred to an operating and control unit, which then automatically makes the settings required for the tool. New business models are also conceivable thanks to tool-specific identification, such as pay per use and the like.

It is also conceivable to store all situations that occur during the use duration of a tool after each use, such as exceeding the permissible speed for the tool, on the identification element and thus to make it easier to investigate the cause in the event of a tool failure. The identification element is preferably an RFID element that can be addressed via NFC, for example.

In another preferred aspect, the sleeve is preferably made of plastic, which ensures the best possible responsiveness of the identification element. Alternatively, the sleeve is made of metal, which additionally increases the protection of the identification element. In order to nevertheless achieve good responsiveness of the identification element, slits are provided in the sleeve, which are optionally sealed with a casting compound or plastic inserts or the like.

In a further preferred aspect, the sleeve has an inner space which is provided and configured to receive an identification element of preferably 1.4 mm×8 mm.

In other words, the sleeve is configured such that the identification element with a diameter between 0.8 mm and 5 mm, preferably a diameter between 1.2 mm and 1.6 mm and in particular a diameter of 1.4 mm is positioned in the inner space enclosed by the sleeve in an assembled state. The length of the identification element is 4 mm to 15 mm, preferably 6 mm to 10 mm and in particular 8 mm. Preferably, the identification element is positioned without play in the inner space of the sleeve.

The identification element is preferably completely housed in the sleeve and thus protected against environmental influences. At the same time, it is ensured that the identification element is not exposed to any driving forces that could damage the identification element. Such a modular design makes it possible to attach the identification element to/in the tool at low cost.

In a further preferred aspect, the orientation portion is oriented in a predetermined manner in relation to the shaft geometry.

In other words, the radial orientation of the orientation portion is matched to the radial orientation of the shaft geometry.

In other words, a center point of the pyramid area of the orientation portion is radially oriented in the same way as the center point of the corresponding trough-shaped depression.

In a further preferred aspect, the predetermined orientation is fixed via a positioning aid between orientation portion and shaft geometry.

In other words, the orientation between the orientation portion and the shaft geometry is defined by a positioning aid, preferably according to a lock-and-key principle or a pin principle. The positioning aid prevents incorrect mounting of the orientation portion in relation to the shaft geometry by an operator.

In a further aspect, there is a medical instrument set comprising at least one tool according to the invention and a handpiece having a drive and a tool chuck, wherein the handpiece is provided and configured to receive the tool in the tool chuck, characterized in that the handpiece comprises a latching and drive unit which axially secures the tool in a state inserted in the handpiece and via which a drive torque is transmitted to the tool.

In other words, there is disclosed a medical instrument set comprising at least one tool according to the disclosure and a handpiece. The handpiece is provided with a drive, wherein the drive is preferably an electric motor or a compressed air turbine. The handpiece has the latching and drive unit, which is provided and configured to latch into the shaft geometry of the tool. In a latched state, the tool is secured axially via the latching and drive unit and the torque generated by the drive is transmitted to the tool via the latching and drive unit.

In a further preferred aspect, the latching and drive unit of the medical instrument set handpiece comprises barrels.

In other words, the latching and drive unit is equipped with latching elements/barrels that latch in the shaft geometry of the tool and via which both the torque is transmitted and the tool is axially secured in the tool. Due to the area support of the barrels in the trough-shaped indentations, a high torque can be transmitted from the latching and drive unit to the tool. The barrels are preferably made from a ceramic material. This enables good torque transmission with low engagement depth of the barrels in the trough-shaped indentations.

In a further preferred aspect, the number of barrels in the handpiece of the medical instrument set corresponds to the number of trough-shaped indentations.

In other words, the latching and drive unit of the handpiece and the tool are matched in such a way that each barrel of the latching and drive unit latches in one of the trough-shaped indentations of the shaft geometry of the tool. This ensures an even torque transmission from the latching and drive unit to the tool.

In a further preferred aspect, the handpiece of the medical instrument set includes a follow element having a negative geometry of the orientation portion and being provided and configured to receive the orientation portion of the tool in a latched state.

In other words, the handpiece of the medical instrument set is equipped with a follow element that is subjected to a spring force in the distal direction via a spring. On a distal front side, the follow element has a geometry that corresponds to the negative geometry of the orientation portion of the tool and holds the tool with the orientation portion.

In a further preferred aspect, the follow element of the handpiece of the medical instrument set is configured such that when the tool is inserted into the handpiece, the follow element orients/positions the tool in a predetermined manner such that the barrels engage the trough-shaped indentations.

In other words, when the tool is inserted into the handpiece, the orientation portion of the tool slides into the negative geometry of the follow element. The follow element is mounted in the handpiece so that it cannot rotate radially and is positioned radially in such a way that the trough-shaped indentations of the shaft geometry, when the tool lies with the orientation portion in the follow element, are oriented radially in accordance with the barrels of the latching and drive unit of the handpiece.

In other words, the follow element ensures that the trough-shaped indentations of the tool are oriented congruently with the barrels of the latching and drive unit of the handpiece in the radial direction before engaging.

The radially positionally correct orientation of the tool or shaft geometry to the latching and drive unit before latching in ensures that the tool can fully latch in, thus preventing damage to the tool or the handpiece. It additionally increases the comfort of the operator, who does not have to adjust the radial orientation of the tool and the handpiece.

In another preferred aspect, the handpiece of the medical instrument set includes an identification unit configured/mounted proximally of the handpiece opening and distally of the latching and drive unit in the shaft of the handpiece.

In other words, the identification unit is configured in the shaft of the handpiece, which reads out the data stored on the identification element of the tool when the tool is inserted. The identification unit is configured between the bearing points of the tool in the shaft of the handpiece.

In yet other words, the identification unit is configured in a distal portion of the handpiece and preferably detects the data stored on the identification element during the process of inserting the tool into the handpiece.

Furthermore, the identification unit may be configured to write data to the identification element when the tool is pulled out of the handpiece.

The identification unit may also be arranged in the handpiece in such a way that the identification element can be read and written during the entire period in which the tool is latched in the handpiece.

### BRIEF DESCRIPTION OF THE DRAWINGS

The disclosure is described in more detail below with reference to the accompanying drawings.

FIG. 1 is a representation of a medical instrument set with a tool according to the invention and a handpiece in a state in which the tool is not inserted into the handpiece.

FIG. 2 is a representation of a sectional view of the medical instrument set with the tool according to the invention and the handpiece in a state in which the tool is inserted into the handpiece.

FIG. 3 are representations of the tool according to the invention with a shaft geometry and an identification element in a sleeve of the first embodiment, which is provided with an orientation portion.

FIG. 4 is a representation of a sectional view of the tool according to the invention and the handpiece in a state in which the tool is inserted into the handpiece and the orientation portion of the tool is not in contact with a follow element of the handpiece.

FIG. 5 is a representation of a sectional view of the tool according to the invention and the handpiece in a state in which the tool is inserted into the handpiece and the orientation portion of the tool is in contact with the follow element of the handpiece.

FIG. 6 is a representation of an enlarged view of a sectional view of the tool according to the invention and the handpiece in a state in which the tool is inserted into the handpiece and the orientation portion of the tool is in contact with the follow element of the handpiece.

FIG. 7 is a representation of a sectional view of the shaft geometry, the identification element and the sleeve with orientation portion of the tool according to the invention in a latched state of the tool in the handpiece.

FIG. 8 is a representation of a sectional view of the tool according to the invention and the handpiece in a latched state.

FIG. 9 shows a representation of a frontal view of the handpiece with a cross-section through the follow element.

FIG. 10 is a representation of a further frontal view of the handpiece with a cross-section through barrels.

FIG. 11 is a representation of a second embodiment of the sleeve, connected to the tool shaft.

FIG. 12 is a representation of a third embodiment of the sleeve, connected to the tool shaft.

### DETAILED DESCRIPTION

In the following, configuration examples of the present disclosure are described on the basis of the associated Figures. In the Figures, identical reference signs denote identical or at least equivalent parts and components. To this extent, a repeated redundant description of such parts and components is expediently dispensed with.

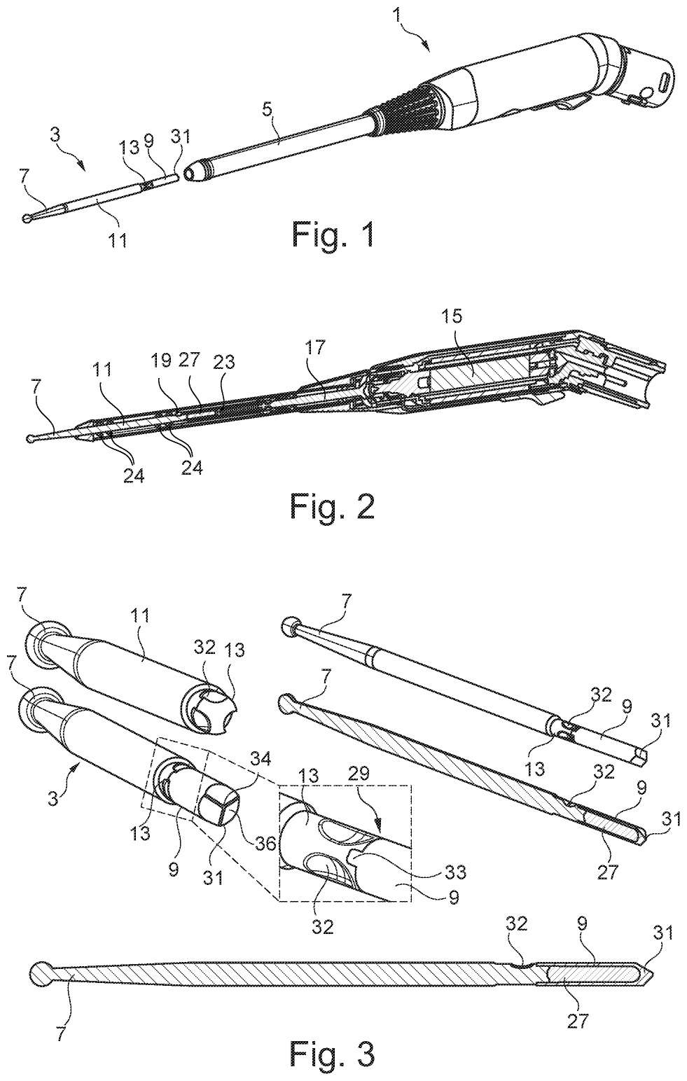

FIG. 1 shows a medical instrument set with a handpiece 1 and a tool 3 according to the invention, which is provided and configured to be inserted into a shaft 5 of the handpiece 1. The tool 3 has a distal working end 7, which forms the actual function of the tool 3 (for example milling, drilling, grinding or smoothing, etc.). On the proximal side of the tool 3 opposite the working end 7, a (separately manufactured) sleeve 9 is provided, which is force-fitted and/or form-fitted and/or firmly bonded to a tool shaft 11. The tool shaft 11 connects the distal working end 7 with the proximal sleeve 9 and is preferably configured in one piece, in particular in one piece of material, with the working end 7. A shaft geometry 13 (exposed radially outward) is configured on/in the tool shaft 11.

FIG. 2 shows a section through the medical instrument set with the handpiece 1 and the tool 3 according to the invention in a state in which the tool 3 is inserted/latched in the handpiece 1.

In the present case, the handpiece 1 includes a drive 15 that is provided and configured to drive the tool 1 (rotationally). The drive 15 is preferably configured as a turbine driven by compressed air, for example, or as an electric motor. The drive 15 exerts a drive torque on a drive shaft 17, which is preferably mounted on rolling or plain bearings. A latching and drive unit 19 (tool holder chuck) is configured on the end portion of the drive shaft 17 opposite the drive 15.

Preferably, a follow element 21 is configured in the latching and drive unit 19, which is subjected to a spring force by a spring 23 in the direction of the drive shaft 17. The follow element 21 is preferably mounted in the latching and drive unit 19 so that it cannot twist/rotate about its longitudinal axis. The shaft geometry 13 of the tool 3 is latched in the latching and drive unit 19 of the handpiece 1 so that the drive torque of the drive 15 can be transmitted to the tool 3. In the latched state, the tool 3 is mounted in the shaft 5 via bearing 24 so that the tool 3 can rotate about its longitudinal axis in the shaft 5 with low loss. Preferably, an identification unit 25 is configured in the shaft between the bearings 24. When the tool 3 is inserted into the shaft 5 of the handpiece 1, the identification unit 25 preferably identifies the tool 3 via an identification element 27, which is preferably configured/housed in the sleeve 9.

The identification element 27 is preferably configured as a wirelessly readable and/or writable storage unit, which stores information that ensures recognition of the tool or of the working end of the tool. In addition, further data and information defined by a user can be stored on the storage unit. Preferably, application-related and/or process-related data such as an article number, a graphic representation of the tool, a LOT specification, a best-before date, use parameters and the like are stored on the storage unit.

The identification element 27 can preferably be read and/or written to wirelessly via Bluetooth and/or NFC or the like.

FIG. 3 shows various views and sections of the tool 3 according to the invention. Accordingly, the tool includes the distal working end 7, the tool shaft 11 with the shaft geometry 13, the proximal sleeve 9 and the identification element 27 in the sleeve 9 as well as an additional (rotational alignment) positioning aid 31, which is preferably arranged/integrated into the sleeve 9 at the proximal end portion of the sleeve 9. Finally, respective orientation profiles 29 are arranged/configured on the proximal end portion of the tool shaft 11 and on the distal end portion of the sleeve that can be brought into operative engagement with each other, whereby the sleeve 9 assumes a predefined relative rotational position to the tool shaft 11 when it is mounted on the tool shaft 11 and thus the proximal (rotational alignment) positioning aid or orientation portion 31 also assumes a predefined relative rotational position to the tool shaft 11.

As already indicated above, the working end 7 is preferably configured in one piece with the tool shaft 11.

The shaft geometry 13 is configured on the lateral surface of the tool shaft 11. The shaft geometry 13 is provided and configured to transmit a torque, which is introduced into the tool 3 via the latching and drive unit 19 of the handpiece 1, and at the same time to secure the tool 3 axially in the handpiece 1. According to the disclosure, the shaft geometry 13 is configured as a trough-shaped or bathtub-shaped depression/indentation 32 in the lateral surface of the tool shaft 11. Preferably, the shaft geometry 13 is configured as a plurality of trough-shaped indentations 32 evenly distributed over the circumference of the tool 3.

The sleeve 9 is provided or configured proximal to the shaft geometry 13. The sleeve 9 is preferably glued, ultrasonically welded, shrink-fitted, soldered or pressed onto the tool shaft 11. The (proximal) front side of the sleeve 9 facing away from the working end 7 forms the positioning aid (orientation portion) 31 and is preferably configured in the shape of a pyramid, with the tip 34 of the pyramid lying on the central fiber of the tool and the pyramid sides 36 being radially aligned in such a way that each pyramid side 36 faces one of the trough-shaped indentations 32.

This predetermined/positionally correct orientation of the orientation portion 31 to the shaft geometry 13 is determined by the positioning aid 29. The positioning aid 29 is preferably configured as a pin 33 or as a pin on the sleeve 9, which fits into a corresponding negative form in the tool shaft 11 or the shaft geometry 13, and thus orients the sleeve 9 and the orientation portion 31 radially positionally correct to the shaft geometry 13.

The sleeve 9 is provided and configured to house the identification element 27. Preferably, the identification element 27 has a smaller diameter than the tool shaft 11. In particular, the diameter of the identification element 27 is preferably less than or equal to 1.4 mm. Furthermore, the axial length of the identification element 27 is preferably 8 mm. Preferably, the sleeve 9 is configured such that in a state in which the sleeve 9 and the tool shaft 11 are connected, the identification element 27 is form-fittingly fixed in the sleeve 9.

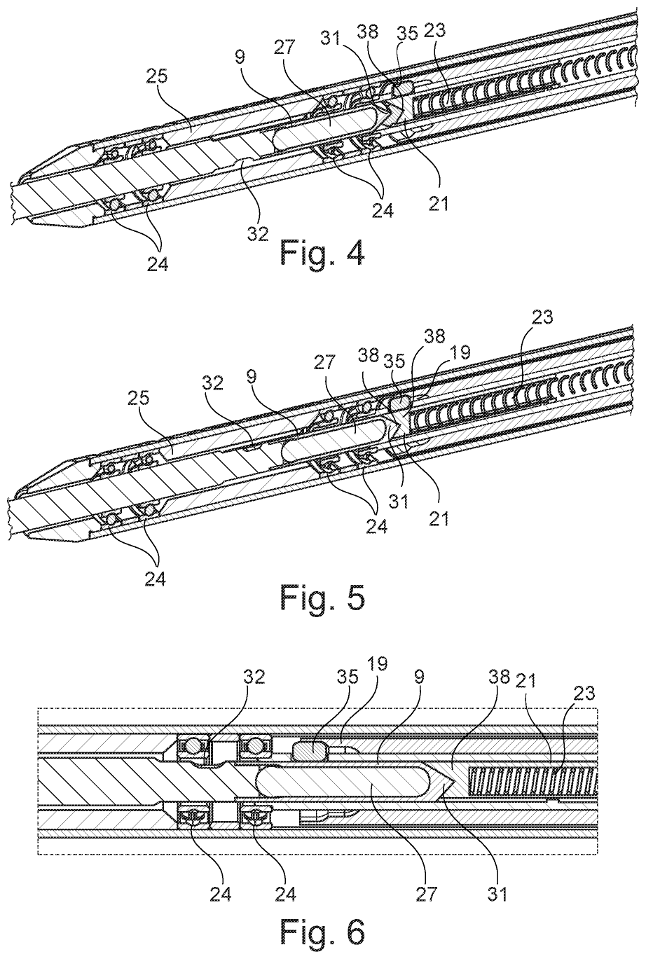

The insertion of the tool 3 into the handpiece 1 is described below with reference to FIG. 4 to FIG. 8.

FIG. 4 shows a section through the medical instrument set in a state in which the tool 3 is inserted into the shaft 5 of the handpiece 1 in such a way that the shaft geometry 13 is not positionally correctly aligned with the latching and drive unit 19. In other words, the tool 3 is oriented in such a way that the trough-shaped indentations of the shaft geometry 13 are not radially aligned with the latching elements/barrels 35 of the latching and drive unit 19. The orientation portion 31 is not in contact with the follow element 21. The follow element 21 has an orientation geometry 38 on a front side facing the tool 3, which corresponds to the negative geometry of the orientation portion 31. This orientation geometry 38 is aligned so that it cannot rotate axially, so that the surfaces of the orientation geometry 38 correspond in their radial arrangement to the radial arrangement of the latching elements/barrels 35 of the latching and drive unit 19.

In other words, the orientation geometry 38 of the follow element 21 is a negative pyramid shape that matches the pyramid shape of the orientation portion 31 of the tool 3 in a key-lock principle. The side surfaces of the negative pyramid shape are radially aligned so that their orientation corresponds to the radial orientation of the latching elements/barrels 35 of the latching and drive unit 19.

During the insertion of the tool 3 into the shaft 5 of the handpiece 1, the identification element 27 positioned in the sleeve 9 passes the identification unit 25 arranged in the shaft, which wirelessly reads out the information stored on the identification element 27.

FIG. 5 shows a section through the medical instrument set in a state in which the tool 3 is inserted slightly further into the shaft 5 of the handpiece 1 than in FIG. 4. During insertion, the orientation portion 31 comes into contact with the follow element 21 on its front side. As the follow element 21 is configured to be radially fixed against rotation, the tool rotates around its longitudinal axis until the side surfaces of the pyramid of the orientation portion 31 are radially aligned with the side surfaces of the negative pyramid of the orientation geometry 38 of the follow element 21. The orientation portion 31 of the tool and the orientation geometry 38 of the follow element 21 are then in surface contact and the trough-shaped indentations 32 of the shaft geometry 13 are radially aligned in accordance with the radial arrangement of the latching elements/barrels 35 of the latching and drive unit 19.

FIG. 6 shows a section through the medical instrument set in a state in which the tool 3 is inserted slightly further into the shaft 5 of the handpiece 1 than in FIG. 5. The spring 23, which applies the spring force to the follow element 21 in the direction of the drive shaft 17, is compressed by the operator by the insertion of the tool 3 into the shaft 5.

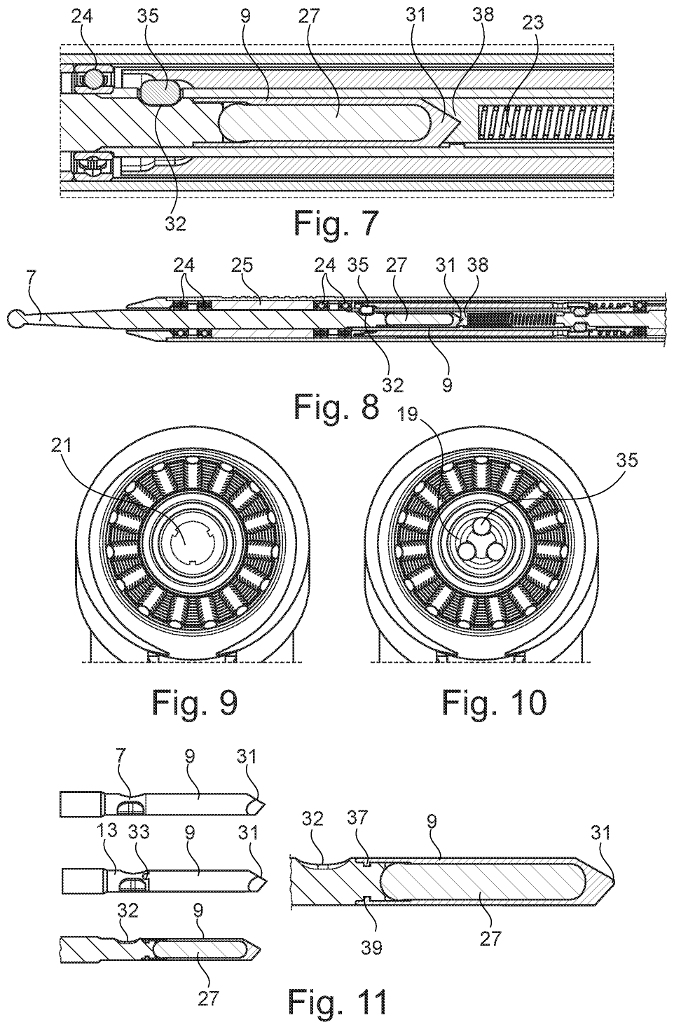

FIG. 7 shows a section through the medical instrument set in a state in which the tool 3 is inserted into the shaft 5 of the handpiece 1 to such an extent that the tool 3 is latched in. In other words, a latching element/barrel 35 of the latching and drive unit 19 is located in each of the trough-shaped indentations 32 of the shaft geometry 13 of the tool 3 in a latched state. Due to the engagement of the latching and drive unit 19 of the handpiece in the shaft geometry 13 of the tool 3, torque introduction from the drive 15 of the handpiece 1 into the tool 3 as well as the axial securing of the tool 3 in the handpiece 1 is implemented.

FIG. 8 also shows a section through the medical instrument set in a state in which the tool 3 is latched in the handpiece 1. The spring 23 is compressed and the tool is rotatable around the center fiber of the tool in the bearings 24.

FIG. 9 is a frontal view of the handpiece 1, in which the follow element 21 is shown cut, in particular to show an anti-rotation device between the follow element 21 and the latching and drive unit 19.

FIG. 10 also shows a frontal view of the handpiece 1. A section is shown explicitly, which shows how the latching elements/barrels 35 of the latching and drive unit 19 engage in the trough-shaped indentations 32 of the shaft geometry 13. The drive torque is transmitted via the latching elements/barrels 35 in particular to the flanks of the trough-shaped indentations 32 of the shaft geometry 13.

FIG. 11 shows an alternative embodiment of the attachment of the sleeve 9 to the tool shaft 11. At an opening portion of the sleeve 9, the sleeve 9 has a latching contour 37 oriented towards the central fiber of the sleeve 9, which in an assembled state engages in a preferably circumferential cut-in 39 on the tool shaft 11. The predetermined orientation is also preferably ensured in this embodiment via the pin 33.

Alternatively, an asymmetrical cut-in 39 and a correspondingly asymmetrically configured latching contour 37 can be used to define a predetermined orientation of the sleeve 9 in relation to the tool shaft 11.

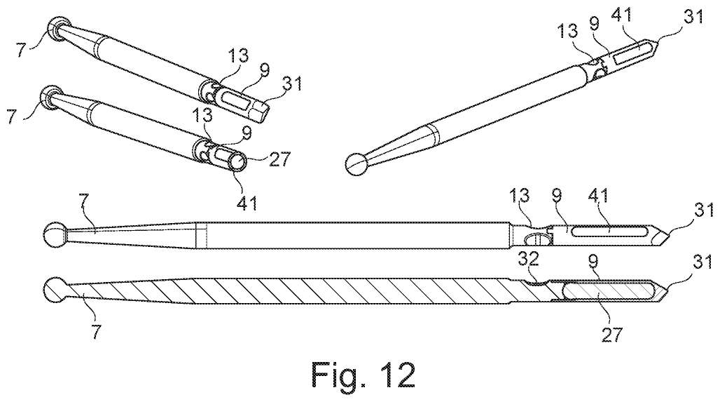

FIG. 12 shows a further alternative embodiment of the sleeve 9. In this embodiment, the sleeve 9 is preferably made of metal and has slits 41 distributed radially around the circumference of the sleeve 9. The slits 41 are preferably sealed with a casting compound or with plastic inserts in order to ensure good responsiveness of the identification element 27. Preferably, the number of slits 41 in the sleeve 9 is between 1 and 10, in particular preferably between 1 and 3.

1.-15. (canceled)

16. A medical tool comprising:
a tool shaft having a shaft region with a shaft geometry;
an orientation portion arranged proximally to the shaft geometry; and
an identification element arranged in a sleeve containing the orientation portion,
the shaft geometry being configured for introducing torque and axial securement,
the shaft geometry comprising at least one trough-shaped indentation in an outer lateral surface of the tool shaft.

17. The medical tool according to claim 16, wherein the at least one trough-shaped indentation consists of a plurality of trough-shaped indentations distributed evenly over a circumference of the medical tool.

18. The medical tool according to claim 16, wherein the orientation portion has a pyramid-shaped geometry.

19. The medical tool according to claim 18, wherein:
the shaft geometry comprises X trough-shaped indentations and the pyramid-shaped geometry comprises Y sides, and
wherein X is an integer multiple of Y.

20. The medical tool according to claim 16, wherein the sleeve is formed as an integral piece of the medical tool.

21. The medical tool according to claim 16, wherein the sleeve is frictionally and/or positively connected to the tool shaft.

22. The medical tool according to claim 16, wherein the orientation portion has a predetermined orientation relative to the shaft geometry, and the predetermined orientation is fixed via a positioning aid between the orientation portion and shaft geometry.

23. The medical tool according to claim 16, wherein the sleeve is made of plastic.

24. The medical tool according to claim 16, wherein slots are provided in the sleeve.

25. A medical instrument set comprising:
at least one medical tool according to one of claim 16; and
a handpiece having a drive and a tool chuck,
wherein the handpiece is configured to receive the at least one medical tool, and
wherein the handpiece comprises a latching and drive unit that axially secures the at least one medical tool in a state inserted in the handpiece and via which a drive torque is transmitted to the at least one medical tool.

26. The medical instrument set according to claim 25, wherein the latching and drive unit comprises barrels.

27. The medical instrument set according to claim 26, wherein:
the at least one medical tool comprises X trough-shaped indentations,
the handpiece comprises Z barrels in the handpiece, and
X=Z.

28. The medical instrument set according to claim 26, wherein the handpiece includes a follow element having a negative geometry of the orientation portion and being provided and configured to receive the orientation portion of the at least one medical tool in a latched state.

29. The medical instrument set according to claim 28, wherein the follow element orients/positions the at least one medical tool in a predetermined manner when the at least one medical tool is inserted into the handpiece such that the barrels engage the trough-shaped indentations.

30. The medical instrument set according to claim 25, wherein the handpiece includes an identification unit configured/mounted proximally of a handpiece opening and distally of the latching and drive unit.
